(19) United States
(12) Patent Application Publication
(10) Pub. No.: US 2013/0090303 A1
(43) Pub. Date: Apr. 11, 2013

(54) Agonists of PY2Y Receptor as a Treatment for Aortic Stenosis and Cardiovascular Calcification

(71) Applicants: Patrick Mathieu, Lac Beauport (CA); Nancy Côté, Quebec (CA)

(72) Inventors: Patrick Mathieu, Lac Beauport (CA); Nancy Côté, Quebec (CA)

(73) Assignee: UNIVERSITE LAVAL, Quebec (CA)

(21) Appl. No.: 13/613,741

(22) Filed: Sep. 13, 2012

Related U.S. Application Data
(60) Provisional application No. 61/534,037, filed on Sep. 13, 2011.

(30) Foreign Application Priority Data
Jun. 4, 2012 (CA) 2,779,085

(51) Int. Cl. A61K 31/7068

(52) U.S. Cl. CPC A61K 31/7068
USPC 514/49; 435/7.1; 435/6.12; 435/29

(57) ABSTRACT

The present invention relates to the treatment and/or prevention of aortic valve stenosis (AVS) and valve mineralization. Particularly, the invention provides a target for intervention in the treatment or prevention of AVS through the administration of the P2Y₂ receptor agonists. Also, the invention provides means to treat hypertension related to decreased arterial compliance and vascular calcification.

### CROSS REFERENCES TO RELATED APPLICATIONS

This application claims priority from U.S. Provisional Application Ser. No. 61/534,037 filed on Sep. 13, 2011, and Canadian Application No. 2,779,085 filed on Jun. 4, 2012, each of which is incorporated herein by reference in its entirety.

The present invention relates to the treatment and/or prevention of aortic valve stenosis (AVS) or calcific aortic valve disease (CAVD). Particularly, the invention provides a target for intervention in the treatment or prevention of AVS through the administration of the P2Y₂ receptor agonists. Also, the invention provides means to treat hypertension related to decreased arterial compliance and vascular calcification.

### BACKGROUND OF THE INVENTION

Calcific aortic valve disease (CAVD) or aortic stenosis (AS) is the most common heart valve disorder. The pathobiological process of AS rely largely on a progressive calcification of the aortic valve. Risk factors for AS includes: age, hypertension, diabetes, male gender and dyslipidemia. There is so far no medical treatment for AS that could prevent the progression of the pathological process once established. It is estimated that AS is present in 2-4% of a population over 65 years of age. Once the process is severe and accompanied by symptoms the only possible cure is an aortic valve replacement (AVR). In North America alone, 100,000 AVR are performed annually. It is worth to emphasize that this figure represents just the tip of the iceberg, since the vast majority of patients have a mild-to-moderate AS, which is prone to progression in a matter of years. Hence, it is possible to envision that if a medical treatment was available it could be possible to stop the disease process and prevent AVR for a large number of afflicted patients.

Progressive mineralization of the aortic valve has been ascribed to an active process, which rely on cellular transformation upon different signals. To this effect, it is suspected that oxidized lipids as well as mechanical stress may participate to the mineralization of the aortic valve. On this score, patients with a bicuspid aortic valve (BAV) have high rate of AS occurring at early age (usually in the 5^{th} decade). BAV have a higher mechanical stress, which may exacerbate a predisposition to calcify valvular tissue. Several mechanisms may be at play during pathological calcification of the aortic valve, but the major determinants remain to be elucidated. To this effect, recent studies indicate that the fate of valvular interstitial cells (VICs), the main cellular component of the aortic valve, largely determines the occurrence of pathological calcification. Studies have highlighted that during calcification VICs are progressively modified toward osteoblastic-like cells, which promote mineralization. The fate of cells, including VICs, depends on signals that are delivered via different types of receptor. To this effect, we have identified that signaling via P2Y₂ receptor in VICs prevent calcification and may thus represent a potential form of medical treatment for AS, CAVD as well as for vascular calcification.

Purinergic system: In the pericellular environment, ATP is liberated by different cellular populations including VICs. ATP then acts upon different types of receptors and may instruct cells for different functions. Thus, depending on signals VICs may enter into an osteoblastic program and promote calcification of the aortic valve. To this effect ATP may modulate the expression of proteins involved into the mineralization process. Purinergic receptors are divided into two broad categories: the ion channel receptors (P2X) and the G-protein-coupled receptor (P2Y). Upon activation P2X receptor promotes the opening of cation channels, which results in changes of electrical potential and the activation of a signaling cascade. Currently, 7 different P2X receptor subunits have been described (P2X₁₋₇) and their expression have been documented in neuronal cells, T cells, macrophages and endothelial cells. On the other hand, 8 different P2Y receptors have been cloned P2Y_{(1,2,4,6,11,12,13,14)} and their expression has been documented in several cell types including VSMCs (vascular smooth muscle cells) and VICs. P2Y receptors have been shown to signal through phospholipase C (PLC), PKC, PI3K and ERK½, which are incidentally pathways that have been previously found implicated in the mineralization of VSMCs/VICs.¹ P2X receptors are stimulated by ATP, whereas P2Y receptors are stimulated by ATP, ADP, UTP and UDP. Studies have found that ATP is released by cells and delivers autocrine and/or paracrine signals important for cellular fate.² Therefore, it is possible that the purinergic system represents an important pathway controlling the mineralization of the aortic valve.

### SUMMARY OF THE INVENTION

In the present work we present evidence that agonists of P2Y₂ receptor prevent the osteoblastic differentiation of valve interstitial cells (VICs), and in doing so abrogate the mineralization of the aortic valve.

Particularly, we present convincing evidence that agonists of P2Y₂ receptor may be considered as potential pharmaceutical agents for the treatment of aortic stenosis (AS), calcific aortic valve disease (CAVD), valve mineralization and/or vascular calcification.

Particularly, the invention provides, for the first time, that the activity of the P2Y₂ receptor is a key regulator of the calcifying process. The mechanism by which activation of this receptor prevents calcification may be related to the decreased expression of Runx2 and/or osteocalcin.

Particularly, the invention provides means to prevent or treat aortic valve stenosis, valve/vascular calcification by the administration of an agonist of the P2Y₂ receptor in a mammal.

The present invention further provides a method for identifying an inhibitor of aortic valve stenosis or valve/vascular calcification, comprising the steps of: a) contacting P2Y₂ receptor with a potential agonist thereof; and b) measuring P2Y₂ receptor activity; whereby activation of the P2Y₂ receptor is an indication that said compound is a potential inhibitor of aortic valve stenosis or valve/vascular calcification.

The invention further provides a method for preventing or treating aortic valve stenosis or valve/vascular calcification in a mammal, including human, suffering therefrom, comprising the step of administering a pharmaceutically effective amount of a P2Y₂ receptor agonist.

Alternatively, the invention is directed to the use of an agonist of P2Y₂ receptor for the manufacture of a medicament for the prevention or treatment of aortic valve stenosis or valve/vascular calcification in a mammal.

The invention further provides an agonist of the P2Y₂ receptor for use to prevent or treat aortic valve stenosis or valve/vascular calcification in a mammal.

The invention further provides the use of an agonist of the P2Y₂ receptor for preventing or treating aortic valve stenosis or valve/vascular calcification in a mammal.

The invention further provides an assay for identifying a potential agonist of P2Y₂ receptor that may be useful for the prevention or treatment of aortic valve stenosis or valve/vascular calcification in a mammal. Particularly, this assay may take the form of a displacement or a competition assay with a compound known to bind and activate P2Y₂ receptor, preferably previously labeled in order to make it easily detectable.

The invention further provides an assay for identifying an agonist of P2Y₂ receptor, comprising the steps of:
a) contacting P2Y₂ receptor or a cell comprising said receptor with a labeled agonist of P2Y₂ receptor to form a P2Y₂ receptor+agonist complex;
b) contacting the P2Y₂ receptor+agonist complex with a test compound;
c) measuring displacement of said labeled agonist from the complex; whereby a displaced labeled agonist is an indication that said test compound may be a potential agonist of P2Y₂ receptor.

The invention further provides an assay for identifying an agonist of P2Y₂ receptor, comprising the steps of:
a) contacting a labeled 2-thio UTP with a test compound to form a 2-thioUTP/compound mixture:
b) contacting the 2-thioUTP/compound mixture with P2Y₂ receptor or a cell comprising P2Y2 receptor;
c) measuring the amount of labeled 2-thioUTP bound to said P2Y₂ receptor; and
d) comparing to amount of labeled 2-thioUTP bound to P2Y₂ receptor in the absence of the test compound;
   wherein a lower amount of labeled 2-thioUTP in step c) compared to step d) is an indication that said test compound may be a potential agonist of P2Y₂ receptor activity.

The invention further provides a functional assay for identifying an agonist of P2Y₂ receptor by way of competing with a purinergic receptor antagonist.

### DETAILED DESCRIPTION OF THE INVENTION

### Abbreviations

AS: aortic stenosis; CAVD: calcific aortic valve disease; VIC: valve interstitial cells; VSMC: vascular smooth muscle cells.

### Definitions

The term “prevent” or “ prevention” as used herein means the prevention of the disease as well as prevention of aggravation of symptoms.

### DESCRIPTION OF THE FIGURES

FIG. 1 represents immunohistochemistry (FIG. 1A); q-PCR analyses (FIG. 1B); and transcript analyses (FIG. 1C) of the P2Y₂ receptor in AS/CAVD valves.

FIG. 2 shows a functional assay with suramin, a purinergic receptor antagonist (FIG. 2A); treatment of isolated cells with 2-thioUTP (a P2Y₂ agonist) (FIG. 2B); and expression levels of bone transcription factor, Runx2 and osteocalcin (FIG. 2C) following treatment with 2-thioUTP.

### DETAILED DESCRIPTION OF PARTICULAR EMBODIMENTS

Particularly, with respect to the different embodiments of the present invention such as methods and use, the mammal is particularly a human.

Particularly, with respect to the methods, use and compounds of the present invention, the test compound is particularly a competitive agonist of P2Y₂ receptor activity, more particularly the agonist compound is an UTP analog. With respect to the method of treatment and use of the invention, most particularly, the agonist is 2-thioUTP.

Particularly, with respect to the assay of the present invention, the 2-thioUTP may be labeled in such as way as to enable its detection by spectroscopic, photochemical, biochemical, immunochemical, chemical, or other physical means.

For example, useful labels include fluorescent dyes, electron-dense reagents, enzyme substrates (e.g., as commonly used in an ELISA), biotin, digoxigenin, co-factors, ligands, chemiluminescent agents, radioisotope, fluorophores, colorimetric haptens, enzymatic label, and combinations thereof or other entities which can be made detectable, such that the compound bound to the label or marker can be determined by detecting the labeled marker compound in a complex. For example, compounds (e.g., label substrates) can be labeled with ¹³¹I, ¹²⁵I, ³⁵S, ¹⁴C, or ³H, either directly or indirectly, and the radioisotope detected by direct counting of radioemission or by scintillation counting. Alternatively, assay components can be enzymatically labeled with, for example, horseradish peroxidase, alkaline phosphatase, beta-galactosidase or luciferase, and the enzymatic label detected by determination of conversion of an appropriate substrate to product. Labels can also be proteins with luminescent properties, e.g., green fluorescent protein and phycoerythrin.

Methods for labeling and guidance in the choice of labels appropriate for various purposes are discussed for example in Sambrook et al. (Molecular Cloning: A Laboratory Manual, Cold Spring Harbor, N.Y., 1989) and Ausubel et al. (In Current Protocols in Molecular Biology, John Wiley & Sons, New York, 1998).

Alternatively, the assay may evaluate the activity of P2Y₂ receptor agonists by way of decreased expression of Runx2 or ostecalcin, particularly in a cell.

The invention further provides an assay for identifying an agonist of P2Y₂ receptor, comprising the steps of:
a) contacting a cell comprising a P2Y₂ receptor with a purinergic receptor antagonist;
b) measuring an amount of calcium in a cell culture from step a) treated with a mineralizing medium;
c) contacting the cell culture from step b) with a test compound; and
d) measuring calcium in cell culture treated in c);
   whereby a decrease in cell culture calcium in d) compared to b) is an indication that said test compound is a potential agonist of P2Y₂ receptor.

### EXAMPLES

### Example 1

### Methods

### Tissues for Analyses and Cell Culture

CAVD valves and control non-calcified valves were obtained. VICs were isolated from control non calcified aortic valves. The protocol was approved by local ethical committee and informed consent has been obtained from the subjects.

### Immunostaining and Histologic Analyses

Immunostaining analyses were performed with the following antibody: P2Y₂ (Santa Cruz Biotechnology, USA). Slides were then incubated with EnVision Dual Link System-HRP, followed by AEC substrate (Dako, Carpinteria, Calif., USA).

### Real-Time PCR

RNA was also extracted from AS valves and cells during in vitro experiments. Total RNA was isolated with RNeasy micro kit from Qiagen (Qiagen, Mississauga, Ont, Canada). The RNA extraction protocol was performed according to manufacturer's instructions using 100 mg of tissue. The quality of total RNA was monitored by capillary electrophoresis (Experion, Biorad, Mississauga, Ont, Canada). Four (4) μg of RNA was reverse transcribed using the Quantitec Reverse Transcription Kit from Qiagen. Quantitative real-time PCR (q-PCR) was performed with Quantitec SYBR Green PCR kit from Qiagen in the Rotor-Gene 6000 system (Corbett Robotics Inc, San Francisco, Calif., USA). The following primers were obtained from (Invitrogen, Burlington, Ont, Canada): P2Y₂, Runx2, osteocalcin. The expression of hypoxanthine-guanine phosphoribosyltransferase (HPRT) (Invitrogen, Burlington, On, Canada) as a reference gene was chosen to normalize the results.

### Determination of Calcium Concentrations

In isolated cells, calcium was measured by the Arsenazo III method (Côte N, et al. J Mol Cell Cardiol 2012;52:1191-202) and results reported as percent changes.

### Valve Interstitial Cells Isolation and In Vitro Analyses of Calcification

Human valve interstitial cells (VICs) were isolated by collagenase digestion. To provoke calcification, cells were incubated for 7 days with a pro-calcifying medium containing: DMEM+5% FBS, 10⁻⁷ M insulin, 50 μg /ml ascorbic acid and NaH₂PO₄ at 2 mM.

### Results

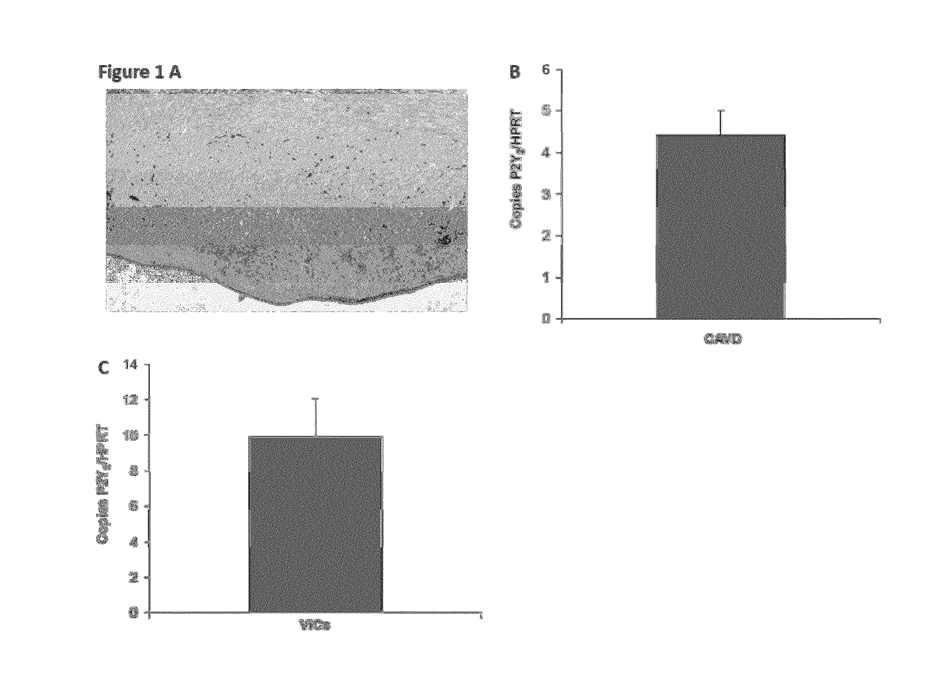

P2Y₂ receptor was documented in AS/CAVD valves by immunohistochemistry, where it was localized near mineralized areas, suggesting that it may have some connections with the calcifying process (FIG. 1A). The presence of P2Y₂ receptor was then confirmed in AS/CAVD valves by q-PCR analyses (FIG. 1B). In isolated human VICs we also found the presence transcripts for P2Y₂ (FIG. 1C).

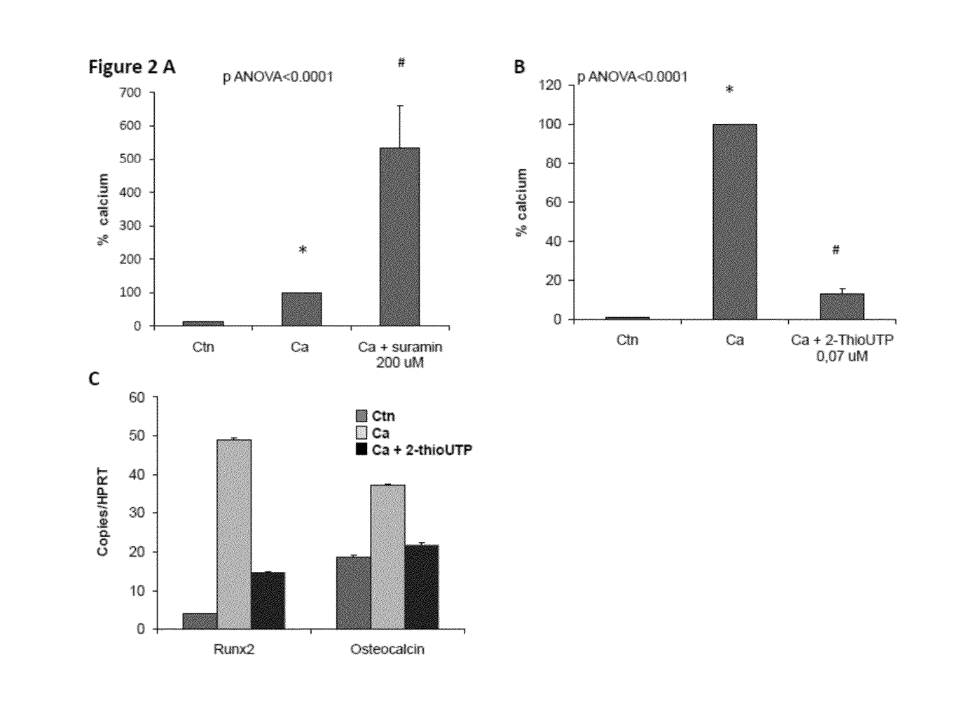

In functional assay, suramin, a purinergic receptor antagonist, increased calcification of isolated VICs suggesting that purinergic receptor(s) are involved in the control of the mineralizing process of the aortic valve (FIG. 2A). On the other hand, a treatment of isolated cells with a P2Y₂ agonist, 2-thioUTP, reduced significantly calcification of isolated VICs (FIG. 2B). Of interest, the treatment with 2-thioUTP decreased the expression of the bone transcription factor, Runx2, as well as its downstream target, osteocalcin (FIG. 2C). When taken together, these facts indicate that P2Y₂ may represent an important target in the treatment of AS.

### Discussion

Calcific aortic valve disease (CAVD) is the most frequent heart valve disorder. It is a progressive disorder related to the mineralization of the valvular tissue.³ In this regard, progressive mineralization of the aortic valve causes in the initial stages no obstruction to the blood flow, and is often referred to as aortic sclerosis.⁴ However, aortic sclerosis may progress to AS, which is the resultant of an ongoing and constant mineralization of the valvular tissue. So far, there is no medical treatment that is available for the treatment of CAVD/AS. In the present work we demonstrate that P2Y₂ receptor is a potential target in the treatment of CAVD/AS and/or cardiovascular calcification. To this effect, 2-thioUTP or other agonist molecules of the P2Y₂ receptor may be used for the treatment of CAVD/AS, valve calcification and/or hypertension caused by vascular calcification.

Mineralization of the aortic valve is an active process. Studies have shown that calcification of the aortic valve is associated with the expression of several osteoblastic genes.³ It should be pointed out that Runx2 is highly expressed during pathological mineralization and is considered as a key transcription factor that mediate osteoblastic differentiation of VICs.¹ Noteworthy, treatment of aortic VICs with P2Y₂ agonist prevented the rise of Runx2 and its downstream target osteocalcin. It is thus likely that stimulation of P2Y₂ receptor in the aortic valve prevents the phenotypic switch of resident cells toward bone-forming cells.

In conclusion, we present herein evidence that 2-thioUTP and other related compounds that are agonists of the P2Y2 receptor are potential pharmaceutical agents that could be used in the treatment and/or prevention of CAVD/AS, valve calcification and/or hypertension caused by vascular calcification.

### REFERENCE LIST

(1) Marie P J. Transcription factors controlling osteoblastogenesis. Arch Biochem Biophys 2008 May 15;473(2):98-105.
(2) Lazarowski E R, Boucher R C, Harden T K. Mechanisms of release of nucleotides and integration of their action as P2X- and P2Y-receptor activating molecules. Mol Pharmacol 2003 October;64(4):785-95.
(3) Rajamannan N M, Subramaniam M, Rickard D et al. Human aortic valve calcification is associated with an osteoblast phenotype. Circulation 2003 May 6;107(17):2181-4.
(4) Freeman R V, Otto C M. Spectrum of calcific aortic valve disease: pathogenesis, disease progression, and treatment strategies. Circulation 2005 Jun. 21;111(24):3316-26.

1. An assay for identifying an agonist of P2Y₂ receptor, comprising the steps of:
a) contacting P2Y₂ receptor or a cell comprising said P2Y₂ receptor with a labeled agonist of P2Y₂ receptor to form a P2Y₂ receptor+labeled agonist complex;
b) contacting the P2Y₂ receptor+labeled agonist complex with a test compound;
c) measuring displacement of said labeled agonist from the complex;
whereby a displaced labeled agonist is an indication that said test compound is a potential agonist of P2Y₂ receptor.

2. The assay of claim 1, wherein said labeled agonist of P2Y₂ receptor is 2-thioUTP.

3. The assay of claim 2, wherein said 2-thioUTP is labeled in such as way as to enable its detection by spectroscopic, photochemical, biochemical, immunochemical, chemical, or other physical means.

4. The assay according to claim 3, wherein said 2-thioUTP is labeled with a label selected from the group consisting of: fluorescent dye, electron-dense reagent, enzyme substrate, biotin, digoxigenin, co-factor, ligand, chemiluminescent agent, radioisotope, fluorophore, colorimetric hapten, enzymatic label, and a combination thereof.

5. The assay of claim 1, wherein said displaced P2Y₂ receptor agonist is selected for further assessing whether it is a compound suitable for the treatment or prevention of aortic stenosis or valve/vascular calcification in a subject.

6. An assay for identifying an agonist of P2Y₂ receptor, comprising the steps of:
a) contacting a labeled 2-thio UTP with a test compound to form a 2-thioUTP/compound mixture:
b) contacting the 2-thioUTP/compound mixture with P2Y₂ receptor or a cell comprising P2Y2 receptor;
c) measuring the amount of labeled 2-thioUTP bound to said P2Y₂ receptor; and
d) comparing to amount of labeled 2-thioUTP bound to P2Y₂ receptor in the absence of the test compound;
wherein a lower amount of labeled 2-thioUTP in step c) compared to step d) is an indication that said test compound is a potential agonist of P2Y₂ receptor activity.

7. The assay according to claim 6, wherein said 2-thioUTP is labeled with a label selected from the group consisting of: fluorescent dye, electron-dense reagent, enzyme substrate, biotin, digoxigenin, co-factor, ligand, chemiluminescent agent, radioisotope, fluorophore, colorimetric hapten, enzymatic label, and a combination thereof.

8. The assay of claim 7, wherein said radioisotope selected from the group consisting of: ¹³¹I, ¹²⁵I, ³⁵S, ¹⁴C, and ³H, and the radioisotope detected by direct counting of radioemission or by scintillation counting.

9. The assay of claim 7, wherein said label is a substrate for an enzyme selected from the group consisting of: horseradish peroxidase, alkaline phosphatase, beta-galactosidase and luciferase, and the enzymatic label is detected by determination of conversion of an appropriate substrate to product.

10. The assay of claim 7, wherein said label is a protein with luminescent properties selected from the group consisting of: green fluorescent protein and phycoerythrin.

11. An assay for identifying a potential compound for the treatment or prevention of aortic stenosis or valve/vascular calcification, comprising the step of:
a) identifying a compound able to decrease expression of Runx2 or ostecalcin in a cell;
b) assessing said compound for its ability to act as an agonist for a purinergic receptor; and
c) selecting said agonist for further assessing whether it is a compound suitable for the treatment or prevention of aortic stenosis or valve/vascular calcification in a subject.

12. An assay for identifying a potential compound for the treatment or prevention of aortic stenosis (AS), calcific aortic valve disease (CAVD), valve mineralization, or hypertension caused by vascular calcification, comprising the step of:
a) identifying a compound able to compete with a purinergic receptor antagonist;
b) assessing said compound for its ability to act as an agonist for said purinergic receptor; and
c) selecting said agonist for further assessing whether it is a compound suitable for the treatment or prevention of aortic stenosis or valve/vascular calcification in a subject.

13. An assay for identifying an agonist of P2Y₂ receptor, comprising the steps of:
a) contacting a cell comprising a P2Y₂ receptor with a purinergic receptor antagonist;
b) measuring an amount of calcium in cell culture from step a), further treated with a mineralizing medium;
c) contacting the cell from step b) with a test compound; and
d) measuring calcium from cell culture treated in c);
whereby a decrease in the level of calcium measured in cell culture in d) compared to b) is an indication that said test compound is a potential agonist of P2Y₂ receptor.

14. The assay of claim 13, further comprising the step of e) selecting said agonist for further assessing whether it is a compound suitable for the treatment or prevention of aortic stenosis or valve/vascular calcification in a subject.

15. A method for preventing or treating aortic valve stenosis (AVS), calcific aortic valve disease (CAVD), valve calcification, or hypertension caused by vascular calcification in a mammal, suffering therefrom, comprising the step of administering a pharmaceutically effective amount of a P2Y₂ receptor agonist.

16. The method according to claim 15, wherein said agonist is a competitive agonist of P2Y₂ receptor activity.

17. The method according to claim 16, wherein said agonist is a UTP analog.

18. The method according to claim 17, wherein the agonist is 2-thioUTP.

19. The method according to claim 15, wherein said mammal is a human.
